(19) United States
(12) Patent Application Publication
(10) Pub. No.: US 2024/0090589 A1
(43) Pub. Date: Mar. 21, 2024

(54) POWER CONTROL SYSTEM

(71) Applicant: Altria Client Services LLC, Richmond, VA (US)

(72) Inventors: Zack W. BLACKMON, Richmond, VA (US); Thomas TRAN, Richmond, VA (US); Terry BACHE, Richmond, VA (US); Jarrett KEEN, Richmond, VA (US); Niall GALLAGHER, Richmond, VA (US); Yannick HOURMAND, Haslingfield (GB); Guillaume MICHEL, Cambridge (GB); Rangaraj S. SUNDAR, Midlothian, VA (US); Eric HAWES, Midlothian, VA (US); Raymond W. LAU, Glen Allen, VA (US)

(73) Assignee: Altria Client Services LLC, Richmond, VA (US)

(21) Appl. No.: 17/947,345

(22) Filed: Sep. 19, 2022

(51) Int. Cl. A24F 40/57; A24F 40/53; A24F 40/60; H02J 7/00

(52) U.S. Cl. CPC A24F 40/57; A24F 40/53; A24F 40/60; H02J 7/007; A61M 15/06

(57) ABSTRACT

A power control system for an aerosol-generating device includes at least one processor and a memory. The memory is coupled to the at least one processor and is configured to store instructions. The at least one processor is configured to execute the instructions to cause the power control system to determine whether a first power to be applied to a heater of the aerosol-generating device exceeds a power threshold, in response to the first power exceeding the power threshold, apply a second power to the heater, and in response to the first power not exceeding the power threshold, apply the first power to the heater. The first power is based on a desired heater temperature. The second power does not exceed the power threshold.

### TECHNICAL FIELD

At least some example embodiments relate to aerosol-generating devices and more particularly, but without limitation, to power control systems for aerosol-generating devices.

### BACKGROUND

Some electronic devices are configured to heat a plant material to a temperature that is sufficient to release constituents of the plant material while keeping the temperature below a combustion point of the plant material so as to avoid any substantial pyrolysis of the plant material. Such devices may be referred to as aerosol-generating devices (e.g., heat-not-burn aerosol-generating devices), and the plant material heated may be tobacco and/or cannabis. In some instances, the plant material may be introduced directly into a heating chamber of an aerosol generating device. In other instances, the plant material may be pre packaged in individual containers to facilitate insertion and removal from an aerosol-generating device.

### BRIEF SUMMARY

New and useful systems, apparatuses, and methods for power control systems for aerosol-generating devices are set forth in the appended claims. Illustrative embodiments are also provided to enable a person skilled in the art to make and use the claimed subject matter.

At least one example embodiment relates to a power control system for an aerosol-generating device. The power control system includes at least one processor and a memory coupled to the at least one processor and storing instructions. The at least one processor is configured to execute the instructions to cause the power control system to determine whether a first power to be applied to the heater of the aerosol-generating device exceeds a power threshold, in response to the first power exceeding the power threshold, apply a second power to the heater, and in response to the first power not exceeding the power threshold, apply the first power to the heater. The first power is based on a required power to reach a desired heater temperature. The second power does not exceed the power threshold.

In at least one example embodiment, the at least one processor is configured to execute the instructions to cause the power control system to detect a start of a session, start a stage timer, increment a power-exceeded variable by a time interval when the first power exceeds the power threshold, monitor the power-exceeded variable against a power-exceeded threshold, and in response to the power-exceeded variable exceeding the power-exceeded threshold, end the session. The stage timer is configured to measure an elapsed time. The power-exceeded variable corresponds to a total amount of time the first power exceeds the power threshold.

In at least one example embodiment, the at least one processor is configured to execute the instructions to cause the power control system to not increment the power-exceeded variable when the aerosol-generating device is operating in a maximum-power phase.

In at least one example embodiment, the at least one processor is configured to execute the instructions to cause the power control system to detect when a puff of the aerosol-generating device is being taken, increment a puff variable when an airflow sensor detects that a puff of the aerosol-generating device has been taken, clear the stage timer, and start the stage timer. The puff variable corresponds to a total number of puffs taken during the session.

In at least one example embodiment, the aerosol-generating device is operating in the maximum-power phase when the elapsed time does not exceed a preheat time threshold and a puff of the aerosol-generating device is not being taken.

In at least one example embodiment, the at least one processor is configured to execute the instructions to vary the power threshold within the session.

In at least one example embodiment, the power threshold is maximized within the session when a puff of the aerosol-generating device is not being taken and the elapsed time does not exceed the preheat time threshold.

In at least one example embodiment, the power threshold is about 10,000 milliwatts when a puff of the aerosol-generating device is not being taken and the elapsed time does not exceed the preheat time threshold.

In at least one example embodiment, the at least one processor is configured to execute the instructions to vary the preheat time threshold within the session, based on the puff variable.

In at least one example embodiment, the preheat time threshold is longer when the puff variable is equal to zero than when the puff variable is greater than zero.

In at least one example embodiment, the preheat time threshold is about 2700 milliseconds.

In at least one example embodiment, the preheat time threshold is about 5200 milliseconds.

In at least one example embodiment, the aerosol-generating device is operating in the maximum-power phase when the elapsed time does not exceed an initial puff time threshold and a puff of the aerosol-generating device is being taken.

In at least one example embodiment, the initial puff time threshold is about 1200 milliseconds.

In at least one example embodiment, at least one processor is configured to execute the instructions to cause the power control system to, detect airflow through the aerosol-generating device, measure a length of time of the airflow through the aerosol-generating device, and determine a puff has occurred based on the length of time.

In at least one example embodiment, the puff length threshold is about 350 milliseconds.

In at least one example embodiment, the session is started when a control button is actuated and the aerosol-generating device begins to preheat.

In at least one example embodiment, the time interval is about 1 millisecond.

In at least one example embodiment, the power-exceeded threshold is about 90 seconds.

In at least one example embodiment, the at least one processor is configured to execute the instructions to cause the power control system to display a fault indicator when the power-exceeded variable exceeds the power-exceeded threshold.

At least one example embodiment relates to a power control system for an aerosol-generating device. The power control system includes at least one processor and a memory coupled to the at least one processor and storing instructions. The at least one processor is configured to execute the instructions to cause the power control system to detect a start of a session, start a stage timer, request a power be applied to a heater of the aerosol-generating device, increment a power-exceeded variable by a time interval when the power exceeds a power threshold, monitor the power-exceeded variable against a power-exceeded threshold, and in response to the power-exceeded variable exceeding the power-exceeded threshold, end the session. The stage timer is configured to measure an elapsed time. The power-exceeded variable corresponds to a total amount of time the power exceeds the power threshold.

One or more example embodiments provide a non-transitory computer-readable storage medium storing computer-readable instructions that, when executed by a controller at a power control system for an aerosol-generating device, cause the controller to perform a method of operating the power control system for the aerosol-generating device, the method comprising: determining whether a first power to be applied to a heater of the aerosol-generating device exceeds a power threshold, in response to the first power exceeding the power threshold, applying a second power to the heater, the second power not exceeding the power threshold, and in response to the first power not exceeding the power threshold, applying the first power to the heater. The first power is based on a desired heater temperature.

One or more example embodiments provide a method of operating a power control system for an aerosol-generating device, the method comprising: determining whether a first power to be applied to a heater of the aerosol-generating device exceeds a power threshold, in response to the first power exceeding the power threshold, applying a second power to the heater, the second power not exceeding the power threshold, and in response to the first power not exceeding the power threshold, applying the first power to the heater. The first power is based on a desired heater temperature.

One or more example embodiments provide a non-transitory computer-readable storage medium storing computer-readable instructions that, when executed by a controller at a power control system for an aerosol-generating device, cause the controller to perform a method of operating the power control system for the aerosol-generating device, the method comprising: detecting a start of a session, starting a stage timer, requesting a power be applied to a heater of the aerosol-generating device, incrementing a power-exceeded variable by a time interval when the power exceeds a power threshold, monitoring the power-exceeded variable against a power-exceeded threshold, and in response to the power-exceeded variable exceeding the power-exceeded threshold, ending the session. The stage timer is configured to measure an elapsed time. The power-exceeded variable corresponding to a total amount of time the power exceeds the power threshold.

One or more example embodiments provide a method of operating a power control system for an aerosol-generating device, the method comprising: detecting a start of a session, starting a stage timer, requesting a power be applied to a heater of the aerosol-generating device, incrementing a power-exceeded variable by a time interval when the power exceeds a power threshold, monitoring the power-exceeded variable against a power-exceeded threshold, and in response to the power-exceeded variable exceeding the power-exceeded threshold, ending the session. The stage timer is configured to measure an elapsed time. The power-exceeded variable corresponding to a total amount of time the power exceeds the power threshold.

Objectives, advantages, and a preferred mode of using the claimed subject matter may be understood best by reference to the accompanying drawings in conjunction with the following detailed description of illustrative embodiments.

### BRIEF DESCRIPTION OF THE DRAWINGS

The various features and advantages of the non-limiting embodiments herein may become more apparent upon review of the detailed description in conjunction with the accompanying drawings. The accompanying drawings are merely provided for illustrative purposes and should not be interpreted to limit the scope of the claims. The accompanying drawings are not to be considered as drawn to scale unless explicitly noted. For purposes of clarity, various dimensions of the drawings may have been exaggerated.

FIG. 1 is a top right, front perspective view of a device in accordance with at least one example embodiment.

FIG. 2A is a top right, front perspective view of the device of FIG. 1, where the lid is opened and where the device includes a capsule.

FIG. 2B is a bottom right, front perspective view of the device of FIG. 1.

FIG. 2C is a bottom view of the device of FIG. 1.

FIG. 3 is a block diagram of a power control system of the device of FIG. 1 in accordance with at least one example embodiment.

FIG. 4 is a block diagram of heating systems of the device of FIG. 1 and the capsule of FIG. 2 in accordance with at least one example embodiment.

FIG. 5 is a timing diagram of operation of the power control system of FIG. 3 in accordance with at least one example embodiment.

FIG. 6 is a block diagram of a method of operating the power control system of FIG. 3 in accordance with at least one example embodiment.

FIG. 7 is a block diagram of another method of operating the power control system of FIG. 3 in accordance with at least one example embodiment.

FIG. 8A is a first portion of a block diagram of another method of operating the power control system of FIG. 3 in accordance with at least one example embodiment.

FIG. 8B is a second portion of the block diagram of the method of operating the power control system of FIG. 3 in accordance with at least one example embodiment.

FIG. 9 is a block diagram of a method of operating a power control system FIG. 3 when a capsule has been inserted in a device in accordance with at least one example embodiment.

FIG. 10 is a block diagram of a method of operating the power control system of FIG. 3 when a puff start has been detected in accordance with at least one example embodiment.

FIG. 11 is a block diagram of another method of operating the power control system of FIG. 3 in accordance with at least one example embodiment.

### DESCRIPTION OF EXAMPLE EMBODIMENTS

Some detailed example embodiments are disclosed herein. However, specific structural and functional details disclosed herein are merely representative for purposes of describing example embodiments. Example embodiments may, however, be embodied in many alternate forms and should not be construed as limited to only the example embodiments set forth herein.

Accordingly, while example embodiments are capable of various modifications and alternative forms, example embodiments thereof are shown by way of example in the drawings and will herein be described in detail. It should be understood, however, that there is no intent to limit example embodiments to the particular forms disclosed, but to the contrary, example embodiments are to cover all modifications, equivalents, and alternatives falling within the scope of example embodiments. Like numbers refer to like elements throughout the description of the figures.

It should be understood that when an element or layer is referred to as being “on,” “connected to,” “coupled to,” or “covering” another element or layer, it may be directly on, connected to, coupled to, or covering the other element or layer or intervening elements or layers may be present. In contrast, when an element is referred to as being “directly on,” “directly connected to,” or “directly coupled to” another element or layer, there are no intervening elements or layers present. As used herein, the term “and/or” includes any and all combinations of one or more of the associated listed items.

It should be understood that, although the terms first, second, third, etc. may be used herein to describe various elements, regions, layers and/or sections, these elements, regions, layers, and/or sections should not be limited by these terms. These terms are only used to distinguish one element, region, layer, or section from another region, layer, or section. Thus, a first element, component, region, layer, or section discussed below could be termed a second element, region, layer, or section without departing from the teachings of example embodiments.

Spatially relative terms (e.g., “beneath,” “below,” “lower,” “above,” “upper,” and the like) may be used herein for ease of description to describe one element or feature's relationship to another element(s) or feature(s) as illustrated in the figures. It should be understood that the spatially relative terms are intended to encompass different orientations of the device in use or operation in addition to the orientation depicted in the figures. For example, if the device in the figures is turned over, elements described as “below”, or “beneath” other elements or features would then be oriented “above” the other elements or features. Thus, the term “below” may encompass both an orientation of above and below. The device may be otherwise oriented (rotated 90 degrees or at other orientations) and the spatially relative descriptors used herein interpreted accordingly.

The terminology used herein is for the purpose of describing various example embodiments only and is not intended to be limiting of example embodiments. As used herein, the singular forms “a,” “an,” and “the” are intended to include the plural forms as well, unless the context clearly indicates otherwise. It will be further understood that the terms “includes,” “including,” “comprises,” and/or “comprising,” specify the presence of stated features, integers, steps, operations, and/or elements, but do not preclude the presence or addition of one or more other features, integers, steps, operations, elements, and/or groups thereof.

When the terms “about” or “substantially” are used in this specification in connection with a numerical value, it is intended that the associated numerical value includes a manufacturing or operational tolerance (e.g., ±10%) around the stated numerical value. Moreover, when the terms “generally” or “substantially” are used in connection with geometric shapes, it is intended that precision of the geometric shape is not required but that latitude for the shape is within the scope of the disclosure. Furthermore, regardless of whether numerical values or shapes are modified as “about,” “generally,” or “substantially,” it will be understood that these values and shapes should be construed as including a manufacturing or operational tolerance (e.g., ±10%) around the stated numerical values or shapes.

Unless otherwise defined, all terms (including technical and scientific terms) used herein have the same meaning as commonly understood by one of ordinary skill in the art to which example embodiments belong. It will be further understood that terms, including those defined in commonly used dictionaries, should be interpreted as having a meaning that is consistent with their meaning in the context of the relevant art and will not be interpreted in an idealized or overly formal sense unless expressly so defined herein.

As used herein, “coupled” includes both removably coupled and permanently coupled. For example, when an elastic layer and a support layer are removably coupled to one another, the elastic layer and the support layer can be separated upon the application of sufficient force.

Hardware may be implemented using processing or control circuitry such as, but not limited to, one or more processors, one or more Central Processing Units (CPUs), one or more microcontrollers, one or more arithmetic logic units (ALUs), one or more digital signal processors (DSPs), one or more microcomputers, one or more field programmable gate arrays (FPGAs), one or more System-on-Chips (SoCs), one or more programmable logic units (PLUs), one or more microprocessors, one or more Application Specific Integrated Circuits (ASICs), or any other device or devices capable of responding to and executing instructions in a defined manner.

One or more example embodiments may be described herein, in at least some instances, as being performed by a power control system of an aerosol-generating device including at least one processor and a memory storing computer-executable instructions, wherein the at least one processor is configured to execute the computer-readable instructions to cause the power control system to perform operations of one or more example embodiments. Additionally, the processor, memory and example algorithms, encoded as computer program code, may serve as means for providing or causing performance of operations discussed herein.

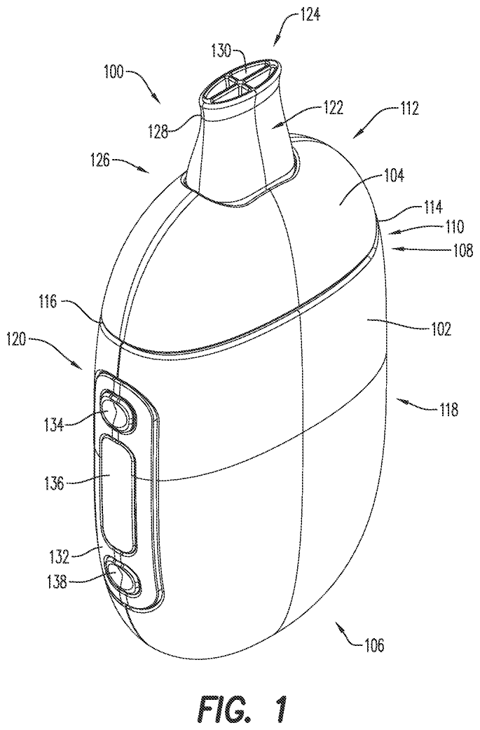

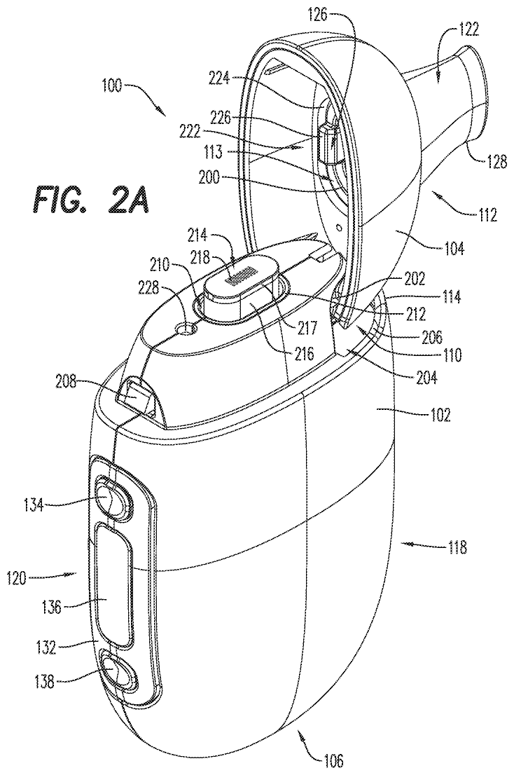

FIGS. 1, 2A, 2B, and 2C are illustrations of a device 100 according to some example embodiments. In some embodiments, the device 100 may be an aerosol-generating device. Referring to FIG. 1, a top perspective view of the device 100 is shown. In some embodiments, a main body of the device 100 may have a general oblong or pebble shape. The main body of the device 100 may include a housing 102 and a lid mechanism or a lid 104. The housing 102 may have a first end 106 and a second end 108 opposite the first end 106. The lid may have a first end 110 and a second end 112 opposite the first end 110. The first end 110 of the lid 104 may be fixedly coupled to the second end 108 of the housing 102 at a first point 114 and releasably couplable to the second end 108 of the housing 102 at a second point 116. The first point 114 of the housing 102 may be on a first side 118 of the device 100. The second point 116 of the housing 102 may be on a second side 120 of the device 100.

In some example embodiments, the device 100 may further include a mouthpiece 122. In at least some example embodiments, the mouthpiece 122 may include a first end 124 and a second end 126 opposite the first end 124. The second end 126 of the mouthpiece 122 may be coupled to the second end 112 of the lid 104. In some embodiments, the second end 126 of the mouthpiece 122 may be releasably coupled to the second end 112 of the lid 104. In at least one example embodiment, the mouthpiece 122 may be tapered between the first end 124 and the second end 126. For example, the diameter or average length/width dimensions of the first end 124 may be smaller than the diameter or average length/width dimensions of the second end 126. Towards the first end 124, the taper may have a slight inward curvature 128 that is configured to receive the lips of an adult consumer and improve the comfort and experience. In some embodiments, the first end 124 may have an oblong or elliptical shape and may include one or more outlets 130. For example, the first end 124 may include four outlets 130, such that four or more different areas or quadrants of the adult consumer's mouth can be engaged during use of the device 100. In other embodiments, the mouthpiece 122 may have fewer outlets than the four outlets 130 or more outlets than the four outlets 130.

In some example embodiments, the housing 102 may include a consumer interface panel 132 disposed on the second side 120 of the device 100. For example, the consumer interface panel 132 may be an oval-shaped panel that runs along the second side 120 of the device 100. The consumer interface panel 132 may include a latch release button 134, as well as a communication screen 136 and/or a control button 138. For example, in at least some example embodiments, the consumer interface panel 132 may include the communication screen 136 disposed between the latch release button 134 and the control button 138. As illustrated, the latch release button 134 may be disposed towards the second end 108 of the device 100, and the control button 138 may be disposed towards the first end 106 of the device 100. The latch release button 134 and the control button 138 may be adult consumer interaction buttons. The latch release button 134 and the control button 138 may have a substantially circular shape with a center depression or dimple configured to direct the pressure applied by the adult consumer, although example embodiments are not limited thereto. The control button 138 may turn on and off the device 100. Though only the two buttons are illustrated, it should be understood more or less buttons may be provided depending on the available features and desired adult consumer interface.

The communication screen 136 may be a consumer interface such as a human-machine interface (HMI) display. In at least one example embodiment, the communication screen 136 may be an integrated thin-film transistor (“TFT”) screen. In other example embodiments, the communication screen 136 is an organic light emitting diode (“OLED”) or light emitting diode (“LED”) screen. The communication screen 136 is configured for adult consumer engagement and may have a generally oblong shape.

In some embodiments, an exterior of the housing 102 and/or the lid 104 may be formed from a metal (such as aluminum, stainless steel, and the like); an aesthetic, food contact rated plastic (such as, a polycarbonate (PC), acrylonitrile butadiene styrene (ABS) material, liquid crystalline polymer (LCP), a copolyester plastic, or any other suitable polymer and/or plastic); or any combination thereof. The mouthpiece 122 may be similarly formed from a metal (such as aluminum, stainless steel, and the like); an aesthetic, food contact rated plastic (such as, a polycarbonate (PC), acrylonitrile butadiene styrene (ABS) material, liquid crystalline polymer (LCP), a copolyester plastic, or any other suitable polymer and/or plastic); and/or plant-based materials (such as wood, bamboo, and the like). One or more interior surfaces or the housing 102 and/or the lid 104 may be formed from or coated with a high temperature plastic (such as, polyetheretherketone (PEEK), liquid crystal polymer (LCP), or the like).

FIG. 2A shows another top perspective view of the device 100 with the lid 104 in an open configuration. The lid 104 may be fixedly coupled to the housing 102 at the first point 114 by a hinge 202, or other similar connector, that allows the lid 104 to move (e.g., swing and rotate) from an open position to a closed position. In some embodiments, the hinge 202 may be a torsion spring. In at least some example embodiments, the housing 102 may include a recess 204 at the first point 114. The recess 204 may be configured to receive a portion of the lid 104 so as to allow for an easy and smooth movement of the lid 104 from the open position to the closed position (and vice versa). The recess 204 may have a structure that corresponds with a relative portion of the lid 104. For example, as illustrated, the recess 204 may include a substantially curved portion 206 that has a general concave shape that corresponds with the curvature of the lid 104, which has a general convex shape.

The lid 104 may be releasably couplable to the housing 102 at the second point 116 by a latch 208, or other similar connector, that allows the lid 104 to be fixed or secured in the closed position and easily releasable to allow the lid 104 to move from the closed position to the open position. In at least one example embodiment, the latch 208 may be coupled to a latch release mechanism disposed within the housing. The latch release mechanism may be configured to move the latch 208 from a first or closed position to a second or open position.

When the lid 104 is in the open position as shown in FIG. 2A, a capsule receiving cavity 210 of the housing 102 is exposed. A capsule connector 212 may define the capsule receiving cavity 210 of the housing 102. In some embodiments, the capsule connector 212 may be mounted or otherwise secured to a printed circuit board (PCB) within the housing 102.

As shown in FIG. 2A, a capsule 214 may be received by the capsule receiving cavity 210. The capsule may house a consumable of the device 100. In some embodiments, not pictured herein, there may be a gasket disposed around the capsule 214 to help secure the capsule 214 in place within the housing 102. The capsule 214 may include a housing 216 configured to contain an aerosol-forming substrate and a heater. In some embodiments, the housing 216 may be in the form of a cover such as a shell or a box sleeve. In some embodiments, the capsule 214 can include a first end cap 217 and a second end cap. The second end cap may be opposite the first end cap 217 such that is disposed within the housing 102 when the capsule 214 is received by the capsule receiving cavity 210.

As discussed herein, an aerosol-forming substrate is a material or combination of materials that may yield an aerosol. An aerosol relates to the matter generated or output by the devices disclosed, claimed, and equivalents thereof. The material may include a compound (e.g., nicotine, cannabinoid), wherein an aerosol including the compound is produced when the material is heated. The heating may be below the combustion temperature so as to produce an aerosol without involving a substantial pyrolysis of the aerosol-forming substrate or the substantial generation of combustion byproducts (if any). Thus, in an example embodiment, pyrolysis does not occur during the heating and resulting production of aerosol. In other instances, there may be some pyrolysis and combustion byproducts, but the extent may be considered relatively minor and/or merely incidental.

The aerosol-forming substrate may be a fibrous material. For instance, the fibrous material may be a botanical material. The fibrous material is configured to release a compound when heated. The compound may be a naturally occurring constituent of the fibrous material. For instance, the fibrous material may be plant material such as tobacco, and the compound released may be nicotine. The term “tobacco” includes any tobacco plant material including tobacco leaf, tobacco plug, reconstituted tobacco, compressed tobacco, shaped tobacco, or powder tobacco, and combinations thereof from one or more species of tobacco plants, such as Nicotiana rustica and Nicotiana tabacum.

In some example embodiments, the tobacco material may include material from any member of the genus Nicotiana. In addition, the tobacco material may include a blend of two or more different tobacco varieties. Examples of suitable types of tobacco materials that may be used include, but are not limited to, flue-cured tobacco, Burley tobacco, Dark tobacco, Maryland tobacco, Oriental tobacco, rare tobacco, specialty tobacco, blends thereof, and the like. The tobacco material may be provided in any suitable form, including, but not limited to, tobacco lamina, processed tobacco materials, such as volume expanded or puffed tobacco, processed tobacco stems, such as cut-rolled or cut-puffed stems, reconstituted tobacco materials, blends thereof, and the like. In some example embodiments, the tobacco material is in the form of a substantially dry tobacco mass. Furthermore, in some instances, the tobacco material may be mixed and/or combined with at least one of propylene glycol, glycerin, sub-combinations thereof, or combinations thereof.

The compound may also be a naturally occurring constituent of a medicinal plant that has a medically-accepted therapeutic effect. For instance, the medicinal plant may be a cannabis plant, and the compound may be a cannabinoid. Cannabinoids interact with receptors in the body to produce a wide range of effects. As a result, cannabinoids have been used for a variety of medicinal purposes (e.g., treatment of pain, nausea, epilepsy, psychiatric disorders). The fibrous material may include the leaf and/or flower material from one or more species of cannabis plants such as Cannabis sativa, Cannabis indica, and Cannabis ruderalis. In some instances, the fibrous material is a mixture of 60-80% (e.g., 70%) Cannabis sativa and 20-40% (e.g., 30%) Cannabis indica.

Examples of cannabinoids include tetrahydrocannabinolic acid (THCA), tetrahydrocannabinol (THC), cannabidiolic acid (CBDA), cannabidiol (CBD), cannabinol (CBN), cannabicyclol (CBL), cannabichromene (CBC), and cannabigerol (CBG). Tetrahydrocannabinolic acid (THCA) is a precursor of tetrahydrocannabinol (THC), while cannabidiolic acid (CBDA) is precursor of cannabidiol (CBD). Tetrahydrocannabinolic acid (THCA) and cannabidiolic acid (CBDA) may be converted to tetrahydrocannabinol (THC) and cannabidiol (CBD), respectively, via heating. In an example embodiment, heat from a heater may cause decarboxylation so as to convert the tetrahydrocannabinolic acid (THCA) in the capsule to tetrahydrocannabinol (THC), and/or to convert the cannabidiolic acid (CBDA) in the capsule to cannabidiol (CBD).

In instances where both tetrahydrocannabinolic acid (THCA) and tetrahydrocannabinol (THC) are present in the capsule, the decarboxylation and resulting conversion will cause a decrease in tetrahydrocannabinolic acid (THCA) and an increase in tetrahydrocannabinol (THC). At least 50% (e.g., at least 87%) of the tetrahydrocannabinolic acid (THCA) may be converted to tetrahydrocannabinol (THC) during the heating of the capsule. Similarly, in instances where both cannabidiolic acid (CBDA) and cannabidiol (CBD) are present in the capsule, the decarboxylation and resulting conversion will cause a decrease in cannabidiolic acid (CBDA) and an increase in cannabidiol (CBD). At least 50% (e.g., at least 87%) of the cannabidiolic acid (CBDA) may be converted to cannabidiol (CBD) during the heating of the capsule.

Furthermore, the compound may be or may additionally include a non-naturally occurring additive that is subsequently introduced into the fibrous material. In one instance, the fibrous material may include at least one of cotton, polyethylene, polyester, rayon, combinations thereof, or the like (e.g., in a form of a gauze). In another instance, the fibrous material may be a cellulose material (e.g., non-tobacco and/or non-cannabis material). In either instance, the compound introduced may include nicotine, cannabinoids, and/or flavorants. The flavorants may be from natural sources, such as plant extracts (e.g., tobacco extract, cannabis extract), and/or artificial sources. In yet another instance, when the fibrous material includes tobacco and/or cannabis, the compound may be or may additionally include one or more flavorants (e.g., menthol, mint, vanilla). Thus, the compound within the aerosol-forming substrate may include naturally occurring constituents and/or non-naturally occurring additives. In this regard, it should be understood that existing levels of the naturally occurring constituents of the aerosol-forming substrate may be increased through supplementation. For example, the existing levels of nicotine in a quantity of tobacco may be increased through supplementation with an extract containing nicotine. Similarly, the existing levels of one or more cannabinoids in a quantity of cannabis may be increased through supplementation with an extract containing such cannabinoids.

The first end cap 217 can include a first opening 218. In some embodiments, the first opening 218 may be a series of openings disposed through the first end cap 217. Similarly, the second end cap can include a second opening that may be a series of openings in some embodiments. In some embodiments, the first end cap 217 and/or the second end cap may be transparent so as to serve as windows configured to permit a viewing of the contents/components (e.g., aerosol-forming substrate and/or heater) within the capsule 214.

The capsule receiving cavity 210 may have a base that may be inside the housing 102. In some embodiments, the base may include at least one contact point that may be configured to couple to one or more contact points of the capsule 214 when the capsule 214 is received by the capsule receiving cavity 210. When the capsule 214 is inserted into the capsule receiving cavity 210, the weight of the capsule 214 itself may not be sufficient to compress the at least one contact point of the base of the capsule receiving cavity 210. As a result, the capsule 214 may simply rest on exposed pins of the at least one contact point without any compression (or without any significant compression) of electrical contacts of the at least one contact point. Additionally, the weight of the lid 104 itself, when pivoted to transition to a closed position, may not compress the electrical contacts of the at least one contact point to any significant degree and, instead, may simply rest on the capsule 214 in an intermediate, partially open/closed position. In such an instance, a deliberate action (e.g., downward force) to close the lid 104 will cause a surface 220 of the lid 104 to press down onto the capsule 214 to provide the desired seal and also cause the capsule 214 to compress and, thus, fully engage the electrical contacts of the at least one contact point.

Additionally, a full closure of the lid 104 may result in an engagement with the latch 208, which may maintain the closed position and the desired mechanical/electrical engagements involving the capsule 214 until released (e.g., via the latch release button 134). The force requirement for closing the lid 104 may help to ensure and/or improve air/aerosol sealing and to provide a more robust electrical connection, as well as improved device and thermal efficiency and battery life by reducing or eliminating early power draws and/or parasitic heating of the capsule 214.

The lid 104 may include an inner cavity 222 that may be adapted to receive the housing 102 when the lid is in the closed position. In some embodiments, the inner cavity 222 of the lid 104 may include an impingement or engagement member or the surface 220 configured to engage the capsule 214 when the lid 104 is pivoted to transition to the closed position. The surface 220 of the lid 104 may include a recess that may correspond to the size and shape of the capsule and/or a resilient material to enhance an interface with the capsule to provide the desired seal. In some embodiments, the lid 104 may further include an opening 224 that may be adapted to receive the second end 126 of the mouthpiece 122. The mouthpiece 122 may include at least one extension 226 that may be received by the opening 224 of the lid 104 to secure the mouthpiece 122 to the lid 104. In some embodiments, the lid 104 may further include a projection that may be configured to couple with a recess 228 of the housing 102. The projection may fit within the recess 228 when the lid 104 is coupled to the housing 102 in the closed position.

During operation of the device 100, power may be applied to a heater within the capsule 214. As a result of application of the power, the capsule 214 may be heated to generate an aerosol. The aerosol generated may be drawn from the device 100 via the mouthpiece 122. The power applied to the heater may vary throughout the session in order for the heater to reach a target temperature. The target temperature may be a temperature calculated to provide a consumer of the device 100 with a desirable sensory experience. In other example embodiments, the heater may be external to the capsule 214 and located within the cavity 210 or adjacent the cavity 210.

In at least one example embodiment, illustrated in FIG. 2B, the housing 102 defines a charging connector or port 170. For example, the charging connector 170 may be defined/disposed in a bottom or second end of the housing 102 distal from the capsule receiving cavity 210. The charging connector 170 may be configured to receive an electric current (e.g., via a USB/mini-USB cable) from an external power source so as to charge the power source 150 internal to the aerosol-generating device 100. For example, in at least one example embodiment, such as best illustrated in FIG. 2C, the charging connector 170 may be an assembly defining a cavity 171 that has a projection 175 within the cavity 171. In at least one example embodiment, the projection 175 does not extend beyond the rim of the cavity 171. In addition, the charging connector 170 may also be configured to send data to and/or receive data (e.g., via a USB/mini-USB cable) from another aerosol generating device (e.g., heat not-burn (HNB) aerosol generating device) and/or other electronic device (e.g., phone, tablet, computer, and the like). In at least one embodiment, the device 100 may instead or additionally be configured for wireless communication (e.g., via Bluetooth) with such other aerosol generating devices and/or electronic devices.

In at least one example embodiment, such as best illustrated in FIG. 2C, a protective grille 172 is disposed around the charging connector 170. The protective grille 172 may be configured to help reduce or prevent debris ingress and/or the inadvertent blockage of the incoming airflow. For example, the protective grille 172 may define a plurality of pores 173 along its length or course. As illustrated, the protective grille 172 may have an annular form that surrounds the charging connector 170. In this regard, the pores 173 may also be arranged (e.g., in a serial arrangement) around the charging connector 170. Each of the pores 173 may have an oval or circular shape, although not limited thereto. In at least one example embodiment, the protective grille 172 may include an approved food contact material. For example, the protective grille 172 may include plastic, metal (e.g., stainless steel, aluminum), or any combination thereof. In at least one example embodiment, a surface of the protective grille 172 may be coated, for example with a thin layer of plastic, and/or anodized.

The pores 173 in the protective grille 172 may function as inlets for air drawn into the aerosol-generating device 100. During the operation of the aerosol-generating device 100, ambient air entering through the pores 173 in the protective grille 172 around the charging connector 170 will converge to form a combined flow that then travels to the capsule 214. For example, the pores 173 may be in fluidic communication with the capsule receiving cavity 210. In at least one example embodiment, air may be drawn from the pores 173 and through the capsule receiving cavity 210. For example, air may be drawn through the capsule 214 received by the capsule receiving cavity 210 and out of the replaceable mouthpiece 190.

As should be understood, the device 100 and capsule 214 include additional components (e.g., heater and internal air flow path) such as described in Atty. Docket No. 24000NV-000847-US, entitled “HEAT-NOT-BURN (HNB) AEROSOL-GENERATING DEVICES AND CAPSULES”, filed on the same day herewith and assigned application No. XX/XXX,XXX, the entire contents of which are herein incorporated by reference.

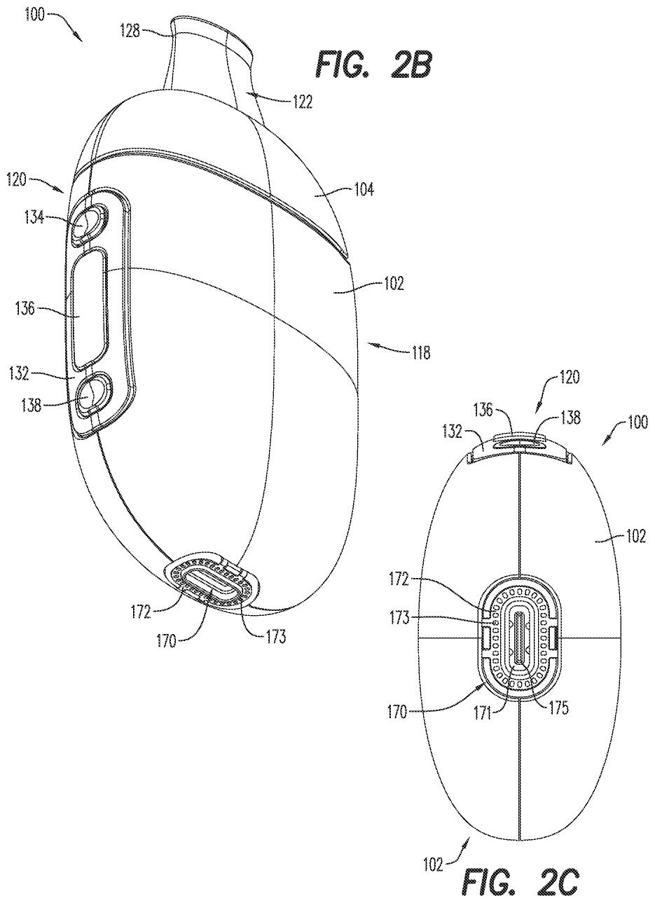

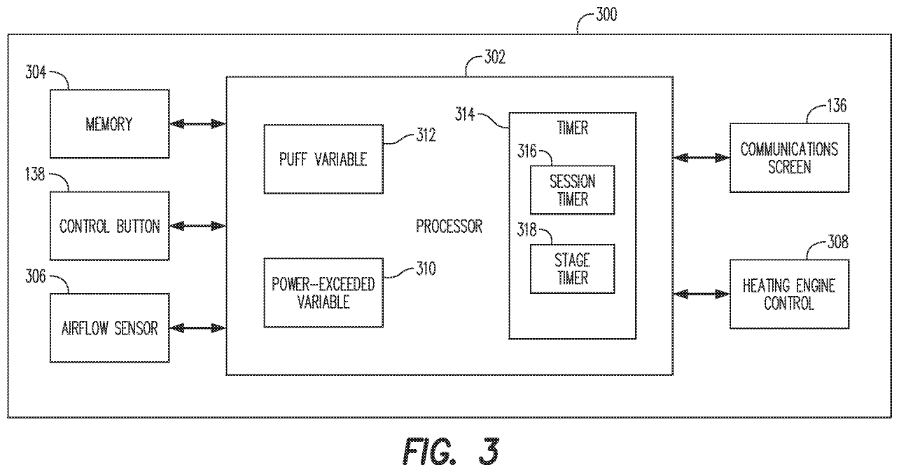

With reference to FIGS. 3 and 4, like reference numerals refer to like elements.

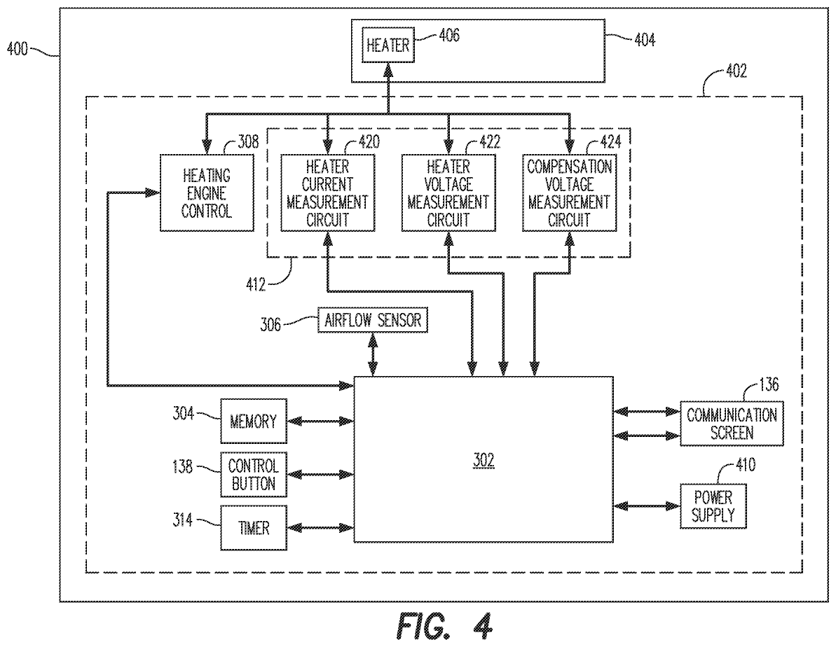

Referring to FIG. 3, a block diagram of a power control system 300 of the device 100 according to an example embodiment is shown. The power control system 300 may be configured to monitor and adjust power applied to the heater of the device 100. The power control system 300 may also be configured to end a session of the device 100 when a power-exceeded threshold is met. The power-exceeded threshold may be met when a power requested to be applied to the heater is above a power threshold for an amount of time which exceeds the power-exceeded threshold. The power threshold is discussed in further detail in reference to FIG. 5 below. The power-exceeded threshold may be about 90 seconds.

When the power-exceeded threshold is met, the power control system 300 may end the session of the device 100. In some embodiments, the power control system 300 may end the session of the device 100 by powering off the heater of the device 100. In some embodiments, the power control system 300 may further be configured to communicate the end of the session of the device 100 to a consumer via the communication screen 136 or another output method of the device 100.

The power control system 300 may include a processor 302, a memory 304, an airflow sensor 306, the communication screen 136, a heating engine control 308, and the control button 138. In some embodiments, the processor 302 may include a power-exceeded variable 310, a puff variable 312, and a timer 314. The processor 302 may communicate with the memory 304, the airflow sensor 306, a heating engine control 308, the communication screen 136, the control button 138, the power-exceeded variable 310, the puff variable 312, and the timer 314.

While the power-exceeded variable 310, the puff variable 312, and the timer 314 are shown within the processor 302, it should be understood that the power-exceeded variable 310, the puff variable 312, and the timer 314 may not be within the processor 302. It should be understood that the values of the power-exceeded variable 310, the puff variable 312, and the timer 314 may be executed/generated by the processor 302 and stored in the memory 304.

The processor 302 may be hardware including logic circuits, a hardware/software combination that may be configured to execute software or a combination thereof. The processor 302 may be configured as a special purpose machine (e.g., a processing device) to execute the software or instructions, stored in the memory 304. The software may be embodied as program code including instructions for performing and/or controlling any or all operations described herein as being performed by the processor 302.

The memory 304 may describe any of the terms “storage medium”, “computer readable storage medium” or “non-transitory computer readable storage medium” and may represent one or more devices for storing data, including read only memory (ROM), random access memory (RAM), magnetic RAM, core memory, magnetic disk storage mediums, optical storage mediums, flash memory devices and/or other tangible machine-readable mediums for storing information. The term “computer-readable medium” may include, but is not limited to, portable or fixed storage devices, optical storage devices, and various other mediums capable of storing, containing, or carrying instructions and/or data. The memory 304 may store operational parameters and computer readable instructions for the processor 302 to perform the algorithms described herein. The memory 304 is illustrated as being external to the processor 302, but in some example embodiments, the memory 304 may be on board the processor 302.

The power-exceeded variable 310 may be a counter that may be set to zero when a session of the device 100 starts. The power-exceeded variable 310 may be incremented by an amount of time that power provided to the heater exceeds the power threshold. The amount of time may be determined by a monitoring performed at a sampling rate by the power control system 300, where the power-exceeded variable 310 increases by a time interval of the sampling rate. In some embodiments, the sampling rate may be about 1 millisecond.

The puff variable 312 may be a counter that may be set to zero when a session of the device 100 starts. The puff variable 312 may be incremented by one each time that a puff is detected. A puff may be detected when a consumer applies a negative pressure through the mouthpiece 122 of the device 100 after placing his or her mouth over the mouthpiece 122 of the device 100. As described in further detail below, in some embodiments, the airflow sensor 306 may be configured to detect a puff such that the puff variable 312 can be incremented each time a consumer takes a puff. The puff variable 312 may be configured to be cleared when a power control system 300 detects the capsule 214 is inserted into the device 100.

The timer 314 may be a timing mechanism, such as an oscillator circuit, to enable the processor 302 to measure times related to use of the device, such as a session time, an elapsed time, a puff time, or the like, of the device 100.

The timer 314 may include one or more timers configured to measure one or more times related to the device 100 and/or the power control system 300. The timer 314 may include a session timer 316 that may be configured to measure the session time. The session time may be a length of a session of the device 100. The session timer 316 may be configured to be cleared when a power control system 300 detects the capsule 214 is inserted into the device 100.

The timer 314 may include a stage timer 318 that may be configured to measure the elapsed time of a stage of the session. The elapsed time may be less than the session time in some embodiments. In some embodiments, the stage timer 318 may be actuated simultaneously with the session timer 316. In some embodiments, the stage timer 318 may be cleared and started throughout the session. In some embodiments, the stage timer 318 is cleared and started when a consumer applies a negative pressure on the device 100 (e.g., a draw or a puff). The stage timer 318 may be configured to be cleared when a power control system 300 detects the capsule 214 is inserted into the device 100.

The airflow sensor 306 may be configured to detect and measure characteristics of airflow through the device 100. For example, the airflow sensor 306 may be configured to detect when air is flowing through the device 100. Additionally, the airflow sensor 306 may be configured to measure a length of time that airflow is flowing through the device 100. In at least one example embodiment, the sensor may be a microelectromechanical system (MEMS) flow or pressure sensor or another type of sensor configured to measure air flow such as a hot-wire anemometer. In an example embodiment, the output of the sensor to measure airflow to the processor 302 is instantaneous measurement of flow (in ml/s or cm3/s) via a digital interface or SPI. In other example embodiments, the sensor may be a hot-wire anemometer, a digital MEMS sensor or other known sensors. The flow sensor may be operated as a puff sensor by detecting a draw when the flow value is greater than or equal to lmL/s, and terminating a draw when the flow value subsequently drops to OmL/s. In an example embodiment, the airflow sensor 306 may be a MEMS flow sensor based differential pressure sensor with the differential pressure (in Pascals) converted to an instantaneous flow reading (in mL/s) using a curve fitting calibration function or a Look Up Table (of flow values for each differential pressure reading). In another example embodiment, the flow sensor may be a capacitive pressure drop sensor.

In some embodiments, the airflow sensor 306 may be communicatively coupled with the puff variable 312 of the processor 302 such that the puff variable 312 is incremented each time the airflow sensor 306 detects that a consumer has started to take a puff of the device 100. In some embodiments, the airflow sensor 306 may be configured to compare the length of time that airflow is flowing through the device 100 to a puff length threshold. If the length of time that airflow is flowing through the device 100 is less than the puff length threshold, the puff variable 312 may not be incremented. If the length of time that airflow is flowing through the device 100 is greater than or equal to the puff length threshold, the puff variable 312 may be incremented.

The communication screen 136 may be configured to display information related to the device 100. The communication screen 136 may be configured to display one or more icons to communicate information related to the device 100. For example, the communication screen 136 may be configured to display a fault indicator that may indicate a fault occurred within the device 100, such as the power-exceeded threshold being met when the power-exceeded variable 310 exceeds the power-exceeded threshold.

The control button 138 may be configured to generate a signal indicating that a consumer has switched the device 100 to an “on” state or to an “off” state. When the device 100 is switched to an “on” state, the device 100 may begin to preheat. In some embodiments, a session may start once the control button 138 is pressed. Once the device 100 is preheated, the session timer 316 may be started in some embodiments.

The heating engine control 308 may be communicatively coupled with the heater of the device 100. The heating engine control 308 may be configured to turn on the heater when the control button 138 detects that the device 100 has been powered on. The heating engine control 308 may additionally be configured to turn off the heater of the device 100 when the power-exceeded threshold has been met and the session of the device 100 is ended.

FIG. 4 illustrates a heating system 400 of the device 100 and the capsule 214 according to one or more example embodiments.

Referring to FIG. 4, the heating system 400 includes a device heating system 402 and a capsule heating system 404. The device heating system 402 may be included in the device 100, and the capsule heating system 404 may be included in the capsule 214.

In the example embodiment shown in FIG. 4, the capsule heating system 404 includes the heater 406.

The capsule heating system 404 may include a body electrical/data interface (not shown) for transferring power and/or data between the device 100 and the capsule 214. According to at least one example embodiment, electrical contacts may serve as the body electrical interface, but example embodiments are not limited thereto.

The device heating system 402 includes the processor 302, a power supply 410, device sensors or measurement circuits 412, the heating engine control 308, the communication screen 136, the control button 138, the memory 304, the timer 314, and the airflow sensor 306. The device heating system 402 may further include a capsule electrical/data interface (not shown) for transferring power and/or data between the device 100 and the capsule 214.

The processor 302 is communicatively coupled to the device sensors 412, the heating engine control 308, the communication screen 136, the memory 304, the control button 138, the timer 314, the airflow sensor 306, and the power supply 410.

The power supply 410 may be an internal power supply to supply power to the device 100 and the capsule 214. The supply of power from the power supply 410 may be controlled by the processor 302 through device power control circuitry (not shown). The power control circuitry may include one or more switches or transistors to regulate power output from the power supply 410. The power supply 410 may be a Lithium-ion battery or a variant thereof (e.g., a Lithium-ion polymer battery).

Still referring to FIG. 4, the device sensors 412 may include a plurality of sensor or measurement circuits configured to provide signals indicative of sensor or measurement information to the processor 302. In the example shown in FIG. 4, the device sensors 412 include a heater current measurement circuit 420, a heater voltage measurement circuit 422, and a compensation voltage measurement circuit 424.

The heater current measurement circuit 420 may be configured to output (e.g., voltage) signals indicative of the current through the heater 406. The heater voltage measurement circuit 422 may be configured to output (e.g., voltage) signals indicative of the voltage across the heater 406. The current and/or the voltage may be used to determine characteristics of the capsule 214, such as temperature. The processor 302 may use the measurements provided by the heater current measurement circuit and the heater voltage measurement circuit to determine the power to be requested to be applied to the heater.

The compensation voltage measurement circuit 424 may be configured to output (e.g., voltage) signals indicative of the resistance of electrical power interface (e.g., electrical connector) between the capsule 214 and the device 100. In some example embodiments, the compensation voltage measurement circuit 424 may provide compensation voltage measurement signals to the processor 302, which may be used to calculate a corrected power to request to be applied to the heater 406.

To measure characteristics and/or parameters of the device 100 and the capsule 214 (e.g., voltage, current, resistance, temperature, or the like, of the heater 406), the processor 302 may sample the output signals from the device sensors 412 at a sampling rate appropriate for the given characteristic and/or parameter being measured by the respective device sensor. In some embodiments, the sampling rate of the output signals from the device sensors 412 may be about 10 milliseconds.

The device heating system 402 may include the airflow sensor 306 to measure airflow through the device 100. The airflow sensor 306 may be configured the same as the airflow sensor 306 is configured as described above with reference to FIG. 3.

Still referring to FIG. 4, the processor 302 may control power to the heater 406 to heat the aerosol-forming substrate in accordance with a heating profile (e.g., heating based on volume, temperature, flavor, or the like). The heating profile may be determined based on empirical data and may be stored in the memory 304 of the device 100.

The heating engine control 308, heater current measurement circuit 420, heater voltage measurement circuit 422 and compensation voltage measurement circuit 424 are further described U.S. application Ser. No. 17/151,375, filed Jan. 18, 2021, the entire contents of which are herein incorporated by reference.

FIG. 5 shows an example manner in which the power threshold varies throughout a session of the device 100 according to one embodiment. FIG. 5 is discussed in further detail below. With regards to the description of FIG. 5, processing circuitry (e.g., the processor 302) may set the various time and power thresholds and apply the power levels (via the heating engine control) to the heater.

The power threshold acts an upper limit for application of the power requested to be applied to the heater. The processor varies the power threshold throughout the session in order to minimize excess power applied at any point during the session while still allowing proper temperature control of the consumable. However, the power threshold may still accommodate minor fluctuations in power to assist in regulating heater temperature. In some embodiments, the power threshold may be configured to vary based on the contents of the consumable.

The power threshold may be determined by the processor 302 based on a phase of the session. The phases of the session include an initial preheat phase, a subsequent preheat phase, an initial puff phase, a final puff phase, a transition phase, and an interpuff phase. In some embodiments, the session may include multiple subsequent preheat phases, multiple initial puff phases, multiple final puff phases, multiple transition phases, and/or multiple interpuff phases. In some embodiments, the initial preheat phase, the subsequent preheat phase, and the initial puff phase may be a maximum-power phase. During a maximum-power phase, the time in which requested power to be applied exceeds the power threshold is not included in the power-exceeded variable 310, as regular use may cause the power to exceed the power threshold during the maximum-power phase. Throughout the specification, a maximum-power phase may be used to describe another phase during which time is not added to the power-exceeded variable 310. Therefore, time during the maximum-power phase does not cause the power-exceeded variable 310 to approach the power-exceeded threshold.

A length of time in which a power threshold is applied may be determined by the processor 302 based on the phase of the session, a time threshold associated with said phase, and/or an action performed by a consumer.

The initial preheat phase occurs during a first application of power by the processor 302 of the session, prior to a puff being taken by a consumer. During the initial preheat phase, the power threshold and a length of the initial preheat phase are configured to allow the heater to heat the consumable to a target temperature. Therefore, the power threshold of the initial preheat phase may be the maximum power threshold throughout the entirety of the session in some embodiments. The power control system 300 may detect the device 100 is operating in the initial preheat phase when the puff variable 312 is zero, a puff is not detected as occurring, and the elapsed time has not exceeded the time threshold. When a negative pressure is not detected by the processor and the puff variable 312 is zero, the time threshold is an initial preheat time threshold T1 in the embodiment shown in FIG. 5. The time threshold T1 may be set by the processor 302 at the start of the preheat and/or may be predetermined based on empirical data. The initial preheat time threshold T1 is a length of time since the start of the session and a maximum length of time of the initial preheat phase. The initial preheat phase may end by a consumer beginning to take a puff of the device 100 or when the initial preheat time threshold T1 is met. In some embodiments, the initial preheat time threshold T1 may be about 5200 milliseconds. In some embodiments, the power threshold T1 during the initial preheat phase (i.e., the initial preheat phase power threshold) may be about 10,000 milliwatts.

The subsequent preheat phase occurs after a puff has been completed. During the subsequent preheat phase, the power threshold and a length of the subsequent preheat phase are configured to allow the heater to reheat the consumable to the target temperature. The processor 302 may detect the device 100 is operating in the subsequent preheat phase when the puff variable 312 is greater than zero, a puff is not detected as occurring, and the elapsed time has not exceeded the time threshold. When a puff is not occurring and the puff variable 312 is greater than zero, the time threshold is a subsequent preheat time threshold T6 in the embodiment shown in FIG. 5. The time threshold T6 may be set by the processor 302 at the start of the preheat and/or may be predetermined based on empirical data. The subsequent preheat time threshold is a length of time measured since the start of the most-recently preceding puff. The subsequent preheat phase may end by a consumer beginning to take a puff of the device 100 or when the subsequent preheat time threshold is met. In some embodiments, the time threshold when a puff is not occurring may be a greater length of time when the puff variable 312 is zero than when the puff variable 312 is greater than zero. In some embodiments, the subsequent preheat time threshold may be about 2700 milliseconds. In some embodiments, the power threshold during the subsequent preheat phase (i.e., the subsequent preheat phase power threshold) may be about 10,000 milliwatts

The initial puff phase and the final puff phase occur when the processor 302 detects that a puff is occurring. During the initial puff phase and the final puff phase, the power threshold is reduced to avoid excess application of energy to air flowing through the device 100.

The processor 302 may detect the device 100 is operating in the initial puff phase when a puff is detected as occurring and the elapsed time has not exceeded the time threshold. When a puff is occurring, the time threshold is an initial puff time threshold, T4 in the embodiment shown in FIG. 5. The initial puff time threshold is a length of time measured since the start of presently occurring puff. In the example embodiment shown in FIG. 5, the puff, and the initial puff phase, begins at T3. The initial puff phase may end by a consumer ending the presently occurring puff of the device 100 or when the initial puff time threshold is met. In some embodiments, the initial puff time threshold is about 1200 milliseconds after a puff begins. In some embodiments, the power threshold during the initial puff phase (i.e., the initial puff phase power threshold) may be about 2000 milliwatts.

The processor 302 may detect the device 100 is operating in the final puff phase when a puff is detected as occurring and the elapsed time has exceeded the time threshold. Because a puff is occurring during the final puff phase, the time threshold is the initial puff time threshold, T4 in the embodiment shown in FIG. 5. The final puff phase may end by a consumer ending the presently occurring puff of the device 100. In the example embodiment shown in FIG. 5, the puff, and the final puff phase, ends at T5. In some embodiments, the power threshold during a final puff phase (i.e., the final puff phase power threshold) may be about 4000 milliwatts.

The transition phase occurs after one of the initial preheat phase and the subsequent preheat phase is completed and a puff is not occurring. During the transition phase, the power threshold is changed by the processor 302 from the power threshold applied during the preceding one of the initial preheat phase and the subsequent preheat phase to the power threshold of the interpuff phase. The power threshold during the transition phase (i.e., the transition phase power threshold) changes linearly over a length of the transition phase. A length of the transition phase is determined by the processor 302 based on whether the transition phase is following the initial preheat phase or the subsequent preheat phase. The processor 302 may detect the device 100 is operating in the transition phase when a puff is not detected as occurring, the elapsed time has exceeded the time threshold of the preceding initial preheat phase or the subsequent preheat phase, and the elapsed time has not exceeded a transition time added to the time threshold of the preceding initial preheat phase or the subsequent preheat phase.

The transition time is a length of time in which the power threshold may transition from a preheat phase (one of the initial preheat phase and the subsequent preheat phase) to an interpuff phase. The transition time begins when the time threshold of the preceding preheat phase concludes due to the time threshold. The transition phase may end when the transition time is reached after conclusion of the preceding preheat phase. The power threshold during the transition phase is changed by the processor 302 based on which percentage of the transition phase is complete. For example, when the transition phase is 50 percent complete (i.e., the transition time is 50 percent complete), the power threshold is 50 percent between the preceding preheat power threshold and the power threshold of the interpuff phase. The transition phase begins when the elapsed time exceeds the time threshold of the preceding preheat phase. The transition time following the initial preheat phase may differ from the transition time following the subsequent preheat phase. An end of the transition time following the initial puff phase is shown as T2 in the embodiment shown in FIG. 5. In some embodiments, the transition time following the initial preheat phase may be about 5000 milliseconds. An end of the transition time following the final puff phase is shown as T5 in the embodiment shown in FIG. 5. In some embodiments, the transition time following the subsequent preheat phase may be about 2500 milliseconds.

The interpuff phase occurs after the transition phase is completed and before the initial puff phase begins. During the interpuff phase, the power threshold is configured to allow the heater to maintain the consumable at a target temperature. The interpuff phase may end when a consumer begins to take a puff of the device 100. The processor 302 may detect the device 100 is operating in the transition phase when a puff is not detected as occurring and the elapsed time has exceeded the transition time added to the time threshold of the preceding initial preheat phase or the subsequent preheat phase. In some embodiments, the power threshold during the interpuff phase (i.e., the interpuff phase power threshold) may be about 4500 milliwatts.

Additionally and alternatively, the power threshold in the interpuff phase may be reduced by the processor 302 based on a length of time measured by the session timer 316. In the embodiment shown in FIG. 5, a second interpuff phase, starting at T7, includes a power threshold which decreases as the session continues. In some embodiments, the power threshold during the interpuff phase will not be reduced when the length of time measured by the session timer 316 is about 19,999 milliseconds or less. In some embodiments, the power threshold during the interpuff phase will be reduced by 25 percent when the length of time measured by the session timer 316 is between about 20,000 milliseconds and about 39,999 milliseconds. For example, in an embodiment in which the power threshold in an interpuff phase is about 2700 milliwatts and the power threshold is reduced by 25 percent due to the length of time measured by the session timer 316, the power threshold would be about 2025 milliwatts. In some embodiments, the power threshold during the interpuff phase will be reduced by 37 percent when the length of time measured by the session timer 316 is between about 40,000 milliseconds and about 194,999 milliseconds. In some embodiments, the power threshold during the interpuff phase will be reduced by half when the length of time measured by the session timer 316 is equal to or greater than about 195,000 milliseconds. In some embodiments, the power threshold during the interpuff phase may be reduced based on a value of the puff variable 312.

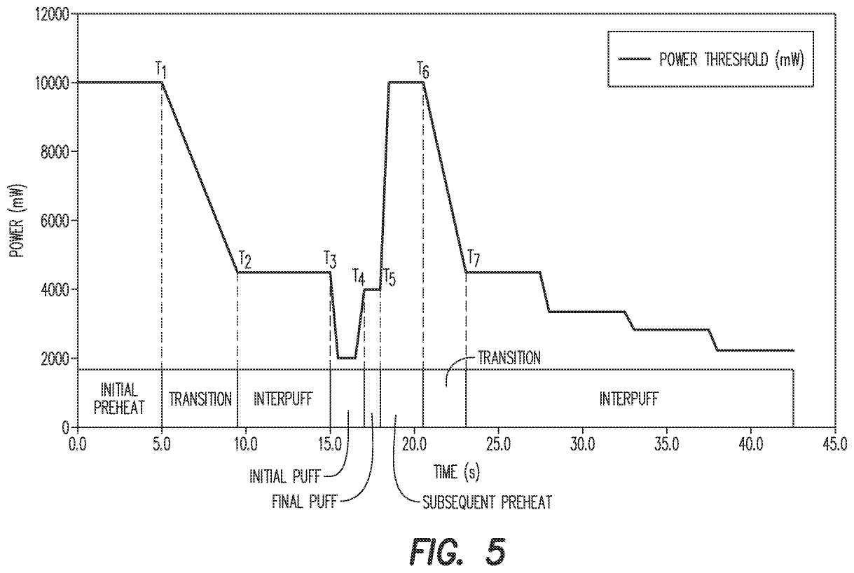

Referring to FIG. 6, a block diagram of a method 600 of operating the power control system 300 of the device 100 is shown. The method 600 of FIG. 6 may be performed by the processor 302, for example. Steps identified as being executed processor 302 in the description below may be executed by other elements of the power control system 300 in some embodiments. For example purposes, the method 600 shown in FIG. 6 will be discussed with regard to the example embodiments shown in FIGS. 3 and/or 4. However, example embodiments should not be limited to these examples.

The method 600 starts at step 602 when the power control system 300 requests a first power be applied to the heater. The first power requested at the step 602 may be a power calculated by the processor 302 to heat the heater to a target temperature, based on the current operating conditions of the device 100.

After the power control system 300 requests for the first power to be applied to the heater at the step 602, the method 600 proceeds to conditional step 604 where the processor 302 determines whether the first power exceeds the power threshold. The method 600 proceeds to step 606 if the first power exceeds the power threshold. At the step 606, the power control system 300 applies a second power to the heater. The second power may be lower than or equal to the power threshold.

If the power control system 300 determines the first power does not exceed the power threshold at the conditional step 604, then the method 600 proceeds to step 608, where the first power is applied to the heater.

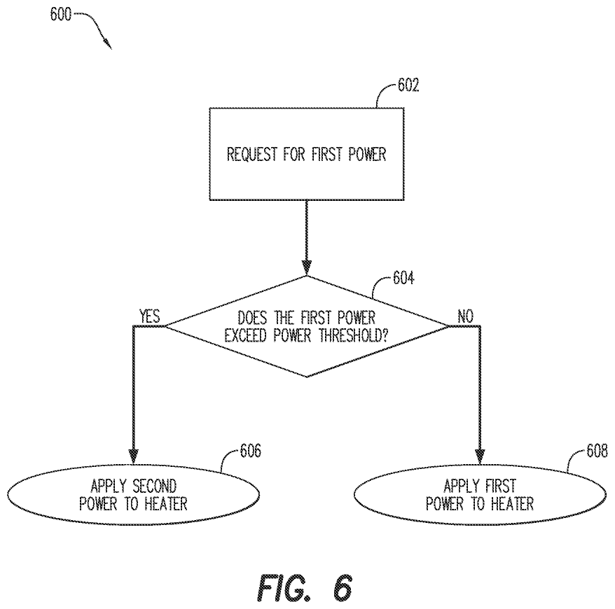

Referring to FIG. 7, a block diagram of a method 700 of operating the power control system 300 of the device is shown. The method 700 of FIG. 7 may be performed by the processor 302, for example. Steps identified as being executed processor 302 in the description below may be executed by other elements of the power control system 300 in some embodiments. For example purposes, the method 700 shown in FIG. 7 will be discussed with regard to the example embodiments shown in FIGS. 3 and/or 4. However, example embodiments should not be limited to these examples.

The method 700 starts at step 702 when a consumer of the device 100 initiates a session of the device. A consumer may initiate a session of the device 100 by actuating a button. At step 704, the processor 302 detects a session has started due to the input by the consumer. Then, at step 705, the power control system 300 begins the stage timer 318.

The method then proceeds to step 706, where the power control system 300 requests a power be applied to the heater. The power requested at the step 706 may be a power calculated by the processor 302 to heat the heater to a target temperature, based on the current operating conditions of the device 100. After power has been requested at the step 706, the method continues to conditional step 708, where the processor 302 determines whether the power requested to be applied to the heater exceeds the power threshold.

If the processor 302 determines at the conditional step 708 that the power requested to be applied exceeds the power threshold, then at step 710, the power control system 300 increments the power-exceeded variable 310. The power-exceeded variable 310 may correspond to a total amount of time the power being requested to be applied to the heater exceeds the power threshold. The power-exceeded variable 310 may be incremented by the time interval of the sampling rate of the power control system 300. The sampling rate of the power control system 300 may be about 1 millisecond. After incrementing the power-exceeded variable 310, the method 700 proceeds to step 712, where the processor 302 determines whether the power-exceeded variable 310 exceeds the power-exceeded threshold. The power-exceeded threshold may be the total amount of time the power requested to be applied to the heater exceeds the power threshold. The power-exceeded threshold may be 90 seconds.

If the processor 302 determines at the conditional step 708 that the power requested to be applied to the heater does not exceed the power threshold, then the method 700 proceeds to the step 712. If the power-exceeded variable 310 exceeds the power-exceeded threshold, then the method 700 proceeds to step 714 and end the session. If the power-exceeded variable 310 does not exceed the power-exceeded threshold, then the method 700 proceeds to step 716 and the processor 302 applies the power to the heater.

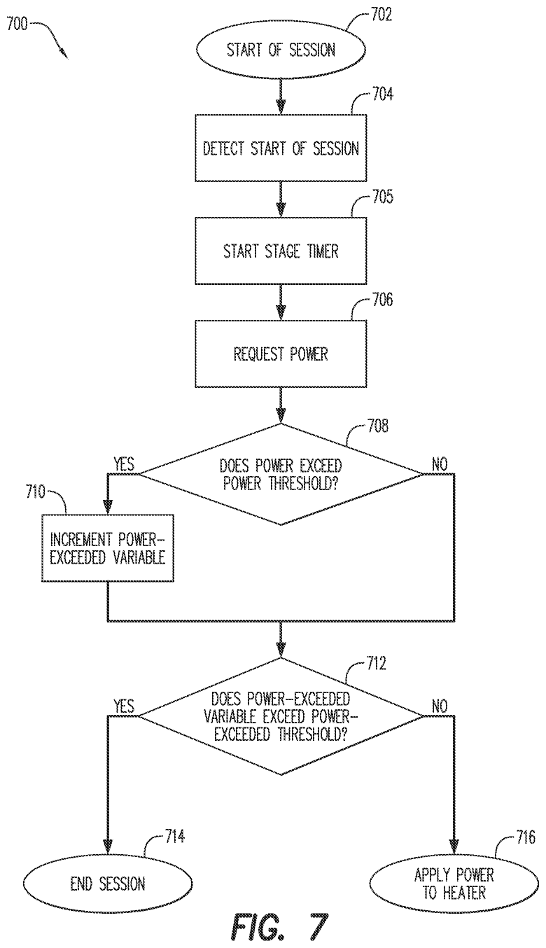

Referring to FIGS. 8A and 8B, a block diagram of a method 800 of operating the power control system 300 of the device is shown. The method 800 of FIGS. 8A and 8B may be performed by the processor 302, for example. Steps identified as being executed processor 302 in the description below may be executed by other elements of the power control system 300 in some embodiments. For example purposes, the method 800 shown in FIGS. 8A and 8B will be discussed with regard to the example embodiments shown in FIGS. 3 and/or 4. However, example embodiments should not be limited to these examples.

The method 800 starts at step 802 when a first power is requested by the processor 302 to be applied to the heater of the device. The first power requested at the step 802 may be a power calculated by the processor 302 to heat the heater to a target temperature, based on the current operating conditions of the device 100.

The method 800 then proceeds to conditional step 804, where the processor 302 determines whether a puff is occurring. In some embodiments, a puff may be detected by the airflow sensor 306. If the processor 302 determines that a puff is occurring at the conditional step 804, then the method 800 continues to step 806, where the processor 302 sets the time threshold to the initial puff time threshold. The method 800 then continues to conditional step 808, where the processor 302 determines whether the elapsed time measured by the stage timer 318 exceeds the time threshold set in the step 806.

If the processor 302 determines that the elapsed time does not exceed the time threshold at the conditional step 808, then the device is operating in the maximum-power phase. and the method 800 continues to step 810, where the processor 302 sets the power threshold to the power threshold of the initial puff phase.

If the processor 302 determines that the elapsed time exceeds the time threshold at the conditional step 808, then the method 800 continues to step 812, where the processor 302 sets the power threshold to the power threshold of the final puff phase.

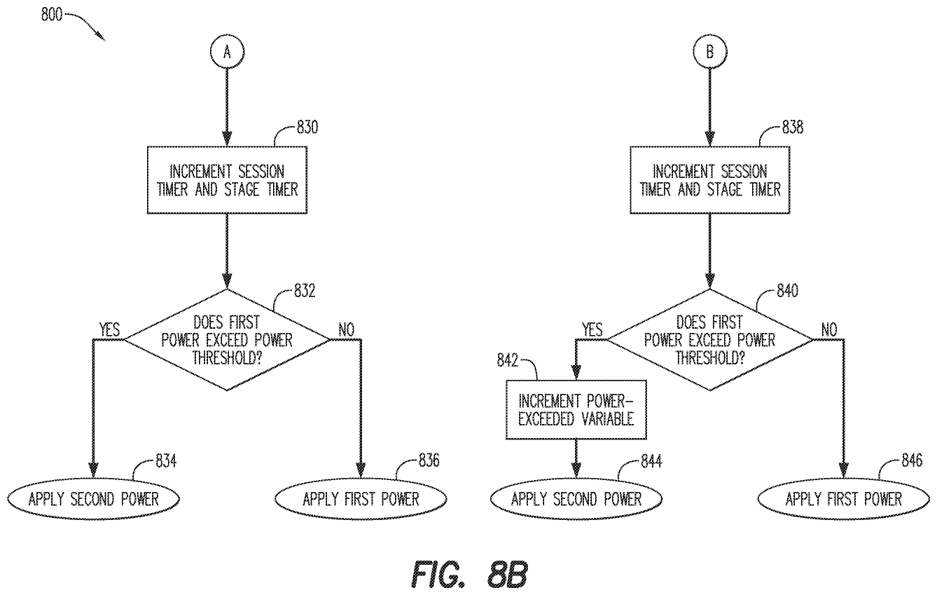

If the processor 302 determines that a puff is not occurring at the conditional step 804, then the method 800 continues to conditional step 814, where the processor 302 determines whether the puff variable 312 is zero. Additional information about the puff variable 312 is described below with reference to FIG. 10.

If the processor 302 determines that the puff variable 312 is not zero at the conditional step 814, then the method 800 proceeds to step 816, where the processor 302 sets the time threshold to the subsequent preheat time threshold. At the step 816, the processor 302 also sets the transition time to the subsequent preheat transition time. Further, at the step 816, the processor 302 sets a preheat power threshold to the power threshold of the subsequent preheat phase.

If the processor 302 determines that the puff variable 312 is zero at the conditional step 814, then the method 800 proceeds to step 818, where the processor 302 sets the time threshold to the initial preheat time threshold. At the step 818, the processor 302 also sets the transition time to the initial preheat transition time. Further, at the step 818, the processor 302 sets the preheat power threshold to the power threshold of the initial preheat phase.

After the step 816 and after the step 818, the method 800 proceeds to conditional step 820. At the conditional step 820, the processor 302 determines whether the elapsed time exceeds the time threshold.

If the processor 302 determines that the elapsed time does not exceed the time threshold at the conditional step 820, then the device is operating in the maximum-power phase. When the processor 302 determines that the elapsed time does not exceed the time threshold at the conditional step 820, the method 800 continues to step 822, where the processor 302 sets the power threshold to the preheat power threshold.

If the processor 302 determines that the elapsed time does exceed the time threshold at the conditional step 820, then the method 800 continues to conditional step 824, where the processor 302 determines whether the elapsed time exceeds a total of the time threshold and the transition time added together.

If the processor 302 determines that the elapsed time does not exceed the sum of the time threshold and the transition time at the conditional step 824, then the method 800 continues to step 826. At the step 826, the processor 302 sets the power threshold to the power threshold of the transition phase, which is based on the transition time, the elapsed time, the time threshold, the preheat power threshold, and the interpuff power threshold.

If the processor 302 determines that the elapsed time exceeds the total of the time threshold and the transition time at the conditional step 824, then the method 800 continues to step 828. At the step 828, the power threshold is set to the power threshold of the interpuff power phase by the processor 302.

After the power threshold is set when the device is operating in the maximum-power phase in the step 810 or the step 822, the method 800 proceeds to step 830, where the processor 302 increments the session timer 316 and the stage timer 318 by the time interval of the sampling rate of the power control system 300.

The method 800 then proceeds from the step 830 to conditional step 832, where the processor 302 determines whether the first power exceeds the power threshold set in either the step 810 or the step 822.

If the processor 302 determines the first power exceeds the power threshold in the conditional step 832, then the method 800 proceeds to step 834. At the step 834, the power control system 300 applies a second power to the heater. The second power may be lower than or equal to the power threshold.

If the power control system 300 determines the first power does not exceed the power threshold in conditional step 832, then the method 800 proceeds to step 836. At the step 836, the power control system 300 applies the first power to the heater.

After the power threshold is set by the processor 302 in the step 812, the step 826, or the step 828, the method 800 proceeds to step 838, where the processor 302 increments the session timer 316 and the stage timer 318 by the time interval of the sampling rate of the processor 302.

The method 800 then proceeds from the step 838 to conditional step 840, where the processor 302 determines whether the first power exceeds the power threshold set in either the step 812, the step 826, or the step 828.

If the processor 302 determines the first power exceeds the power threshold in the conditional step 840, then the method 800 proceeds to step 842. At the step 842, the processor 302 increments the power-exceeded variable 310 by the time interval of the sampling rate of the power control system 300. After the processor 302 increments the power-exceeded variable 310 in the step 842, the method proceeds to step 844. At the step 844, the power control system 300 applies a second power to the heater. The second power may be lower than or equal to the power threshold.

If the processor 302 determines the first power does not exceed the power threshold in conditional step 838, then the method proceeds to step 846. At the step 846, the power control system 300 applies the first power to the heater.

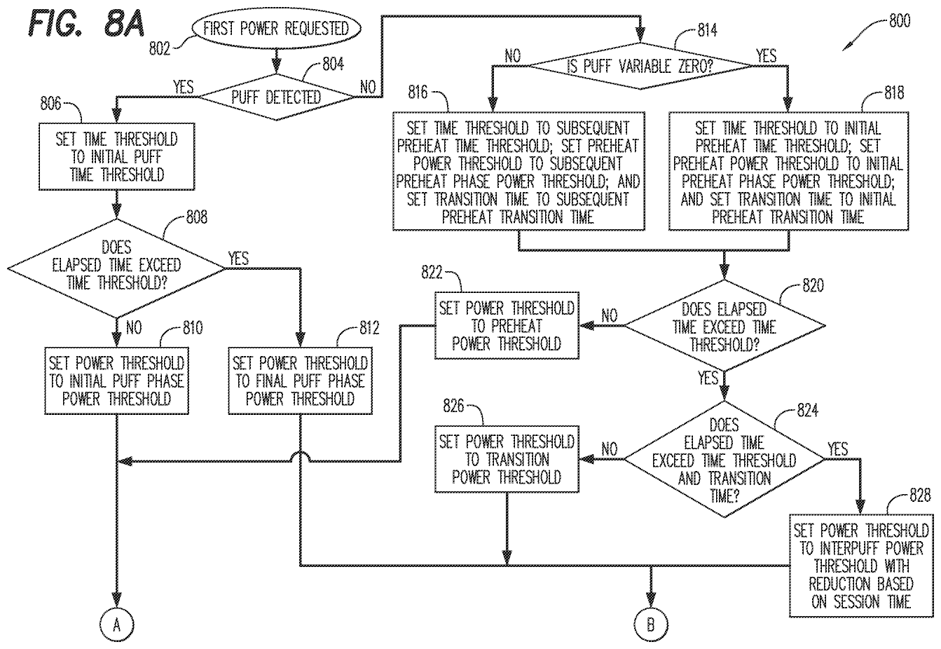

Referring to FIG. 9, a block diagram of a method 900 of resetting the power control system 300 upon insertion of a capsule is shown. The method 900 of FIG. 9 may be performed by the processor 302, for example. Steps identified as being executed processor 302 in the description below may be executed by other elements of the power control system 300 in some embodiments. For example purposes, the method 900 shown in FIG. 9 will be discussed with regard to the example embodiments shown in FIGS. 3 and/or 4. However, example embodiments should not be limited to these examples.

The method 900 starts at step 902 when the processor 302 detects a capsule has been inserted into the device. After insertion of the capsule into the device has been detected by the processor 302 at the step 902, the method 900 proceeds to step 904. At the step 904, the processor 302 clears the stage timer 318 and the puff variable 312 to zero. After the stage timer 318 and the puff variable 312 have been cleared, the method 900 proceeds to step 906. At the step 906, the processor 302 clears the power-exceeded variable 310 to zero. After the power-exceeded variable 310 has been cleared, the method 900 proceeds to step 908. At the step 908, the power control system 300 receives direction from a consumer of the device 100 to start a session of the device 100.

Referring to FIG. 10, a block diagram of a method 1000 of resetting the power control system 300 upon a start of a puff is shown. The method 1000 of FIG. 10 may be performed by the processor 302, for example. Steps identified as being executed processor 302 in the description below may be executed by other elements of the power control system 300 in some embodiments. For example purposes, the method 1000 shown in FIG. 10 will be discussed with regard to the example embodiments shown in FIGS. 3 and/or 4. However, example embodiments should not be limited to these examples.

The method 1000 starts at step 1002 when the power control system 300 detects a consumer has started taking a puff of the device. After the start of a puff of the device has been detected by the power control system 300 at the step 1002, the method 1000 proceeds to step 1004. At the step 1004, the processor 302 clears the stage timer 318 to zero. After the stage timer 318 has been cleared by the processor 302 at the step 1004, the method 1000 proceeds to step 1006. At the step 1006, the processor 302 increments the puff variable 312. After the puff variable 312 has been incremented by the processor 302 at the step 1006, the method 1000 proceeds to step 1008. At the step 1008, the power control system 300 requests a power be applied to the heater.

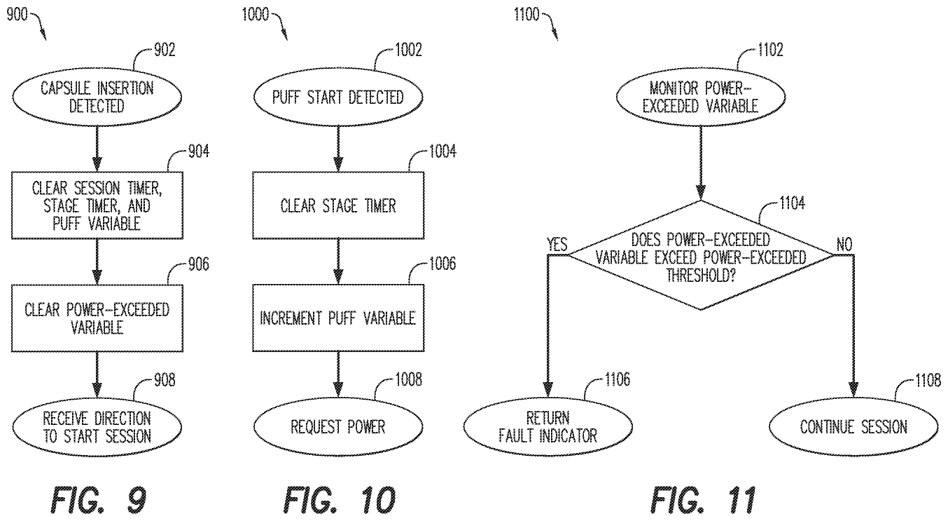

Referring to FIG. 11, a block diagram of a method 1100 of the power control system 300 monitoring the power-exceeded variable 310 against the power-exceeded threshold. The method 1100 of FIG. 11 may be performed by the processor 302, for example. Steps identified as being executed processor 302 in the description below may be executed by other elements of the power control system 300 in some embodiments. For example purposes, the method 1100 shown in FIG. 11 will be discussed with regard to the example embodiments shown in FIGS. 3 and/or 4. However, example embodiments should not be limited to these examples.

The method 1100 starts at step 1102 when the processor 302 monitors the power-exceeded variable 310. This monitoring of the power-exceeded variable 310 may be after incrementing the power-exceeded variable 310.

After the power control system 300 has requested the power be applied at the step 1102, the method 1100 proceeds to conditional step 1104. At the conditional step 1104, the processor 302 determines whether the power-exceeded variable 310 exceeds the power-exceeded threshold.

If the processor 302 determines at the conditional step 1104 that the power-exceeded variable 310 exceeds the power-exceeded threshold, then the method proceeds to step 1106. At the step 1106, the power control system 300 displays a fault indicator on the device.

If the processor 302 determines at the conditional step 1104 that the power-exceeded variable 310 does not exceed the power-exceeded threshold, then the method proceeds to step 1108. At the step 1108, the power control system 300 does not return a fault to the device, allowing the session to continue.

The systems, apparatuses, and methods described herein may provide significant advantages. The power control system 300 may provide a way to prevent excessive power being applied to the heater. The power control system 300 may be configured to power off the heater of the device 100 when the power-exceeded threshold has been exceeded. This may provide a preferrable sensory experience for a consumer. Additionally, the power control system 300 may provide a way to communicate an ending of the session due to excessive power usage to a consumer. For example, the power control system 300 may provide an indication to a consumer of when the power-exceeded threshold has been exceeded by the power-exceeded variable 310 and the session is ending.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. A power control system for an aerosol-generating device, the power control system comprising:
at least one processor; and
a memory coupled to the at least one processor and storing instructions;
wherein the at least one processor is configured to execute the instructions to cause the power control system to,
determine whether a first power to be applied to a heater of the aerosol-generating device exceeds a power threshold, the first power being based on a desired heater temperature,
in response to the first power exceeding the power threshold, apply a second power to the heater, the second power not exceeding the power threshold, and
in response to the first power not exceeding the power threshold, apply the first power to the heater.

2. The power control system of claim 1, wherein the at least one processor is configured to execute the instructions to cause the power control system to:
detect a start of a session,
start a stage timer, the stage timer configured to measure an elapsed time,
increment a power-exceeded variable by a time interval when the first power exceeds the power threshold, the power-exceeded variable corresponding to a total amount of time the first power exceeds the power threshold,
monitor the power-exceeded variable against a power-exceeded threshold, and
in response to the power-exceeded variable exceeding the power-exceeded threshold, end the session.

3. The power control system of claim 2, wherein the at least one processor is configured to execute the instructions to cause the power control system to,
detect when a puff of the aerosol-generating device is being taken,
increment a puff variable when an airflow sensor detects that a puff has been taken, the puff variable corresponding to a total number of puffs taken during the session,
clear the stage timer, and
start the stage timer.

4. The power control system of claim 3, wherein the aerosol-generating device is operating in a maximum-power phase when the elapsed time does not exceed a preheat time threshold and a puff is not being taken.

5. The power control system of claim 4, wherein the at least one processor is configured to execute the instructions to vary the power threshold within the session.

6. The power control system of claim 4, wherein the at least one processor is configured to execute the instructions to vary the preheat time threshold within the session based on the puff variable.

7. The power control system of claim 3, wherein the at least one processor is configured to execute the instructions to cause the power control system to,
detect airflow through the aerosol-generating device,
measure a length of time of the airflow through the aerosol-generating device, and
determine a puff has occurred based on the length of time.

8. The power control system of claim 2, wherein the at least one processor is configured to execute the instructions to cause the power control system to display a fault indicator when the power-exceeded variable exceeds the power-exceeded threshold.

9. The power control system of claim 2, wherein the power threshold varies within the session.

10. The power control system of claim 2, wherein the at least one processor is configured to execute the instructions to cause the power control system to not increment the power-exceeded variable when the aerosol-generating device is operating in a maximum-power phase.

11. A power control system for an aerosol-generating device, the power control system comprising:
at least one processor; and
a memory coupled to the at least one processor and storing instructions;
wherein the at least one processor is configured to execute the instructions to cause the power control system to,
detect a start of a session,
start a stage timer, the stage timer configured to measure an elapsed time,
request a power be applied to a heater of the aerosol-generating device,
increment a power-exceeded variable by a time interval when the power exceeds a power threshold, the power-exceeded variable corresponding to a total amount of time the power exceeds the power threshold,
monitor the power-exceeded variable against a power-exceeded threshold, and
in response to the power-exceeded variable exceeding the power-exceeded threshold, end the session.

12. The power control system of claim 11, wherein the at least one processor is configured to execute the instructions to cause the power control system to,
detect when a puff of the aerosol-generating device is being taken,
increment a puff variable when an airflow sensor detects that a puff has been taken, the puff variable corresponding to a total number of puffs taken during the session,
clear the stage timer, and
start the stage timer.

13. The power control system of claim 12, wherein the aerosol-generating device is operating in a maximum-power phase when the elapsed time does not exceed a preheat time threshold and a puff is not being taken.

14. The power control system of claim 13, wherein the power threshold varies within the session.

15. The power control system of claim 14, wherein the power threshold is maximized within the session when a puff is not being taken and the elapsed time does not exceed the preheat time threshold.

16. The power control system of claim 13, wherein the preheat time threshold varies throughout the session, based on the puff variable.

17. The power control system of claim 16, wherein the preheat time threshold is longer when the puff variable is equal to zero than when the puff variable is greater than zero.

18. The power control system of claim 12, wherein the aerosol-generating device is operating in the maximum-power phase when the elapsed time does not exceed an initial puff time threshold and a puff is being taken.

19. The power control system of claim 12, wherein detecting that a puff is being taken comprises:
detecting, with an airflow sensor, airflow through the aerosol-generating device; and
measuring a length of time of the airflow through the aerosol-generating device,
wherein when the length of time of the airflow through the aerosol-generating device exceeds a puff length threshold, puff is being taken, and
wherein when the length of time of the airflow through the aerosol-generating device does not exceed the puff length threshold, a puff is not being taken.

20. The power control system of claim 11, wherein the session is started when a control button is actuated and the aerosol-generating device begins to preheat.

21. The power control system of claim 11, wherein the at least one processor is configured to execute the instructions to cause the power control system to display a fault indicator when the power-exceeded variable exceeds the power-exceeded threshold.

22. The power control system of claim 11, wherein the power threshold varies within the session.

23. The power control system of claim 11, wherein the at least one processor is configured to execute the instructions to cause the power control system to not increment the power-exceeded variable when the aerosol-generating device is operating in a maximum-power phase.
